(19) United States
(12) Patent Application Publication
(10) Pub. No.: US 2016/0060426 A1
(43) Pub. Date: Mar. 3, 2016

(54) MIXTURES OF EPOXIDIZED FATTY ACID ESTERS

(71) Applicants: Benjamin WOLDT, Bochum (DE); Florian BOECK, Munster (DE); Andreas GEVERS, Bottrop (DE); Michael GRASS, Haltern am See (DE)

(72) Inventors: Benjamin WOLDT, Bochum (DE); Florian BOECK, Munster (DE); Andreas GEVERS, Bottrop (DE); Michael GRASS, Haltern am See (DE)

(73) Assignee: Evonik Degussa GmbH, Essen (DE)

(21) Appl. No.: 14/836,092

(22) Filed: Aug. 26, 2015

(30) Foreign Application Priority Data
Aug. 26, 2014 (EP) 14182190.0

(51) Int. Cl. C08K 5/1515; C08K 5/09; C07D 301/02

(52) U.S. Cl. CPC C08K 5/1515; C07D 301/02; C08K 5/09

(57) ABSTRACT

A mixture (1) can be prepared in a controlled manner and used as a plasticizer, the mixture contains an epoxidized fatty acid ester A; and a compound B containing a fatty acid chain and having no functional group including a multiple bond in the fatty acid chain. The proportion by mass of compound B is smaller in said mixture (1) than a proportion of compound B in a mixture (2) of compounds containing a fatty acid chain from which the mixture (1) comprising epoxidized fatty acid ester A has been prepared.

### BACKGROUND OF THE INVENTION

1. Field of the Invention

The present invention relates to a mixture comprising an epoxidized fatty acid ester, to a process for preparing such mixture, to a composition comprising a corresponding mixture and to the use of the mixture as plasticizer for a polymer.

2. Discussion of the Background

Epoxidized fatty acid esters can be used as plasticizers for polymers, for example PVC. The background art describes, for example in documents WO 01/98404 A2, WO 2011/072346 A1, US 2002/0013396 A1, GB 805,252 B and U.S. Pat. No. 4,486,561 B, the use of epoxidized fatty acid esters of natural origin as plasticizers. Plasticizers employed in these documents are the products that are the direct result of esterification and epoxidation of fatty acids.

The epoxidized fatty acid esters described in document WO 2013/003225 A2, prior to use as plasticizers, are subjected to an additional workup step. For this purpose, the proportion of epoxidized linolenic acid residues is reduced compared to the proportion of epoxidized linoleic acid residues in the mixture of epoxidized fatty acid esters used as plasticizers, which results in a plasticizer having a lower proportion of “impurities”, which can be used in medical products.

### SUMMARY OF THE INVENTION

The problem addressed by the present invention was that of providing novel plasticizers having good, preferably improved, plasticizer properties. Advantageously, these plasticizers should be based on naturally occurring oils.

The problem is solved by mixtures of epoxidized fatty acid esters in which the proportion of the compounds containing fatty acid chains which do not have any functional groups including multiple bonds has been reduced.

The present invention relates to a mixture (1), comprising:
an epoxidized fatty acid ester A; and
a compound B containing a fatty acid chain and having no functional group including a multiple bond in the fatty acid chain;
wherein a proportion by mass of compound B is smaller in said mixture (1) than a proportion of compound B in a mixture (2) of compounds containing a fatty acid chain from which the mixture (1) comprising epoxidized fatty acid ester A has been prepared.

In another embodiment, the present invention relates to a process for preparing a mixture (1) comprising an epoxidized fatty acid ester, comprising:
1) reducing a proportion of a fatty acid or fatty acid ester which does not have any functional group including a multiple bond in the fatty acid chain in a mixture (2) comprising a compound having a fatty acid chain prior to the epoxidation of the fatty acid and/or fatty acid ester;
OR
reducing a proportion of a fatty acid or fatty acid ester which does not have any functional group including a multiple bond in the fatty acid chain in a mixture (2) comprising a compound having a fatty acid chain after the epoxidation of the fatty acid and/or fatty acid ester;
2) expoxidizing said fatty acid and/or said fatty acid ester, to obtain an expoxidized fatty acid and/or an epoxidized fatty acid ester;
3) esterifying the epoxidized fatty acid, to obtain an epoxidized fatty acid ester; and
4) optionally, transesterifying said epoxidized fatty acid ester.

In another embodiment, the present invention relates to a mixture, comprising:

an epoxidized fatty acid ester which has been prepared by the above process.

In another embodiment, the present invention relates to a plasticizer for a polymer, comprising: the above mixture.

Further, the present invention relates to a method for improving the gelation of a plastisol and/or for reducing the viscosity of a plastisol and/or for reducing the glass transition temperature of a composition prepared with the above mixture and/or for improving the thermal stability of a composition prepared with the above mixture,

said method comprising:

incorporating said above mixture in said plastisol and/or said composition.

Moreover, the present invention relates to a composition, comprising:

a mixture as above; and

one or more polymers selected from the group consisting of polyvinyl chloride, a copolymer of vinyl chloride with vinyl acetate or with butyl acrylate, polyalkyl methacrylate (PAMA), polyvinyl butyral (PVB), polyurethane, a polysulphide, polylactic acid (PLA), polyhydroxybutyral (PHB) and nitrocellulose.

### DETAILED DESCRIPTION OF THE INVENTION

Any ranges mentioned herein below include all values and subvalues between the lower and upper limits of said range.

The invention provides a mixture comprising epoxidized fatty acid esters and containing a smaller proportion by mass of compounds containing fatty acid chains and having no functional groups including multiple bonds in the fatty acid chain than the mixture of compounds containing fatty acid chains from which the mixture comprising epoxidized fatty acid esters has been prepared. The proportions by mass are each based on the entirety of all the compounds containing fatty acid chains.

Fatty acid esters are understood in the context of this invention to mean esterification products (esters) of fatty acids with monohydric alcohols. Accordingly, fatty acid esters consist of a fatty acid residue, which is also called fatty acid chain, and an alkoxide residue.

Compounds which contain fatty acid chains and do not contain any further functional groups including multiple bonds in the fatty acid chain (i.e. aside from the ester or acid function) are referred to hereinafter simply as “saturated compounds” or “saturated”.

The mixture comprising epoxidized fatty acid esters according to the above subject-matter is also referred to hereinafter simply as “inventive” mixture or else as “depleted” mixture.

A mixture comprising epoxidized fatty acid esters and containing a comparable (equal) proportion by mass of compounds containing fatty acid chains and having no functional groups including multiple bonds in the fatty acid chain to the mixture of compounds containing fatty acid chains from which the mixture comprising epoxidized fatty acid esters has been prepared is referred to hereinafter as “non-depleted” mixture. The change in the proportions by mass based exclusively on the epoxidation of double bonds is not included in these considerations. For example, a mixture of fatty acid esters containing the same amount of “saturated” compounds as the natural oil from which the mixture has been prepared is a “non-depleted” mixture. A “non-depleted” mixture, in the course of preparation thereof, has not gone through a process step which was designed to reduce the proportion of compounds which contain fatty acid chains and do not have any further functional groups including multiple bonds in the fatty acid chain (aside from the ester or acid function).

Preferably, the mixture of the compounds containing fatty acid chains from which the mixture comprising epoxidized fatty acid esters has been prepared has been obtained from a vegetable oil or a mixture of a plurality of vegetable oils, where the oil or the mixture of oils preferably has an (average) double bond content fatty acid chain—determined by finding the ratios of ¹H NMR signals—in the range from 0.8 to 2.5, preferably from 1.0 to 2.0 and especially from 1.2 to 1.6. The method for determining the double bond content is described in the experimental.

The vegetable oil or the mixture of a plurality of vegetable oils preferably contains one or more oils from the group of soya oil, linseed oil, sunflower oil, safflower oil, rapeseed oil and tall oil.

The mixture of compounds containing fatty acid chains from which the mixture comprising epoxidized fatty acid esters has been prepared is preferably a fatty acid mixture or a fatty acid ester mixture.

If, in one embodiment of the present invention, the mixture of compounds containing fatty acid chains from which the inventive mixture comprising epoxidized fatty acid esters has been prepared is a fatty acid mixture, the inventive mixture comprising epoxidized fatty acid esters contains a smaller proportion by mass of fatty acid esters which do not have any functional groups including multiple bonds in the fatty acid chain than that of fatty acids which do not have any functional groups including multiple bonds in the fatty acid chain that are present in the mixture of fatty acids from which the mixture of epoxidized fatty acid esters has been prepared. The proportions by mass here are always based on the entirety of all the components containing fatty acid chains. Expressed in simplified form: If the inventive mixture comprising epoxidized fatty acid esters has been prepared from fatty acids, the proportion of saturated fatty acid esters in the inventive mixture is smaller than the proportion of saturated fatty acids in the fatty acid mixture used for preparation of the inventive mixture, based in each case on the entirety of all the components containing fatty acid chains.

If, in another embodiment of the present invention, the mixture of compounds containing fatty acid chains from which the inventive mixture comprising epoxidized fatty acid esters has been prepared is a fatty acid ester mixture, the inventive mixture comprising epoxidized fatty acid esters contains a smaller proportion by mass of fatty acid esters which do not have any functional groups including multiple bonds in the fatty acid chain than the mixture of fatty acid esters from which the mixture of epoxidized fatty acid esters has been prepared. The proportions by mass here are always based on the entirety of all the components containing fatty acid chains. Expressed in simplified form: If the inventive mixture comprising epoxidized fatty acid esters has been prepared from fatty acid esters, the proportion of saturated fatty acid esters in the inventive mixture is smaller than the proportion of saturated fatty acid esters in the fatty acid ester mixture used for preparation of the inventive mixture, based in each case on the entirety of all the components containing fatty acid chains.

In a preferred embodiment, the proportion by mass of the compounds containing fatty acid chains and having no functional groups including multiple bonds in the fatty acid chain has been reduced in the inventive mixture comprising epoxidized fatty acid esters by at least 20%, preferably by at least 40%, with preference by at least 60% and especially by at least 80% compared to the mixture of the compounds containing fatty acid chains from which the mixture comprising epoxidized fatty acid esters has been prepared.

The percentage reduction can be calculated as follows:

$$reduction [ \% ] = \frac{\begin{matrix}“ saturated compounds ” in the reactant - \\ “ saturated compounds ” in the inventive mixture\end{matrix}}{” saturated compounds ” in the reactant} \cdot 100$$

where
“saturated compounds” in the reactant=proportion of compounds containing fatty acid chains and containing no further functional groups including multiple bonds in the fatty acid chain (i.e. aside from the ester or acid function) in the mixture of compounds containing fatty acid chains from which the mixture comprising epoxidized fatty acid esters has been prepared in % by mass, based on the entirety of all the products containing fatty acid chains
“saturated compounds” in the inventive mixture=amount of compounds containing fatty acid chains and containing no further functional groups including multiple bonds in the fatty acid chain (i.e. aside from the ester or acid function) in the inventive mixture, based on the entirety of all the products containing fatty acid chains

The present invention preferably provides a mixture comprising epoxidized fatty acid esters in which the proportion by mass of compounds containing fatty acid chains and having no functional groups including multiple bonds in the fatty acid chain has been reduced by at least 20%, preferably by at least 40%, with preference by at least 60% and especially by at least 80% compared to the mixture of the compounds containing fatty acid chains from which the mixture comprising epoxidized fatty acid esters has been prepared.

In the mixture comprising epoxidized fatty acid esters, with preference, the mean number of epoxy groups per fatty acid chain—determined by forming the ratios of ¹H NMR signals—is greater than 1.0, preferably greater than 1.2 and especially greater than 1.4. The method for determining the mean number of epoxy groups is described in the experimental.

If the mixture of the compounds containing fatty acid chains from which the mixture comprising epoxidized fatty acid esters has been prepared has been obtained from linseed oil, the mean number of epoxy groups per fatty acid chain—determined by forming the ratios of NMR signals—is preferably greater than 1.4, more preferably greater than 1.6 and especially greater than 1.8.

The alkoxide residues of the fatty acid esters in the inventive mixture comprising epoxidized fatty acid esters are preferably selected from alkoxide residues containing 1 to 20, preferably 2 to 15 and especially 4 to 11 carbon atoms, where the alkoxide residues preferably (aside from the alkoxide function) do not have any further functional groups including multiple bonds. Preferably, the alkoxide residues of the fatty acid esters in the inventive mixture are selected from methyl, ethyl, propyl, butyl, tert-butyl, iso-butyl, 2-methylbutyl, 3-methylbutyl, n-pentyl, hexyl, heptyl, iso-heptyl, octyl, iso-octyl, 2-ethylhexyl, nonyl, n-nonyl, iso-nonyl, decyl, iso-decyl, 2-propylheptyl, undecyl and tridecyl radicals.

In one embodiment, if the inventive mixture contains epoxidized fatty acid esters of iso-nonanol,
the proportion of derivatives of the alkoxide residue of n-nonanol in the mixture comprising epoxidized iso-nonyl fatty acid esters is less than 2 mol %, based on the entirety of all the epoxidized iso-nonyl fatty acid esters, and/or
the proportion of iso-butene which has been converted in the synthesis of the precursor of the iso-nonanol used in the preparation of the iso-nonyl fatty acid esters, based on the entirety of the butene used for preparation of the iso-nonanol, is greater than 0.5 mol %.

The present invention preferably provides a mixture comprising epoxidized fatty acid esters
in which the proportion by mass of compounds containing fatty acid chains and having no functional groups including multiple bonds in the fatty acid chain has been reduced by at least 20%, preferably by at least 40%, with preference by at least 60% and especially by at least 80% compared to the mixture of the compounds containing fatty acid chains from which the mixture comprising epoxidized fatty acid esters has been prepared and
in which the alkoxide residues of the fatty acid esters are selected from alkoxide residues containing 1 to 20, preferably 2 to 15 and especially 4 to 11 carbon atoms and not having any further functional groups including multiple bonds (aside from the alkoxide function), where, in the case that the mixture comprising epoxidized fatty acid esters comprises epoxidized fatty acid esters of iso-nonanol, the proportion of derivatives of the alkoxide residue of n-nonanol in the mixture comprising epoxidized iso-nonyl fatty acid esters is less than 2 mol %, based on the entirety of all the epoxidized iso-nonyl fatty acid esters, and/or the proportion of iso-butene which has been converted in the synthesis of the precursor of the iso-nonanol used in the preparation of the iso-nonyl fatty acid esters, based on the entirety of the butene used for preparation of the iso-nonanol, is greater than 0.5 mol %.

In one embodiment, the inventive mixture comprising epoxidized fatty acid esters contains less than 90% by mass, less than 75% by mass, less than 50% by mass or no epoxidized fatty acid esters containing iso-nonanol residues, based on the entirety of all the epoxidized fatty acid esters.

Preferably, the alkoxide residues of at least 50% by mass, preferably at least 65% by mass, more preferably at least 85% by mass and especially at least 95% by mass of all the fatty acid esters in the inventive mixture comprising epoxidized fatty acid esters have the same number of carbon atoms.

In another embodiment, two, three, four or more epoxidized fatty acid esters having different alkoxide residues in the epoxidized fatty acid esters are present in the inventive mixture. For example, an inventive mixture may contain 20% to 80% by mass of epoxidized fatty acid esters having 5 carbon atoms in the alkoxide residue and 20% to 80% by mass of epoxidized fatty acid esters having 9 carbon atoms in the alkoxide residue, where the proportions are again based on the entirety of all the components containing fatty acid chains.

With preference, the inventive mixture comprising epoxidized fatty acid esters contains less than 20% by mass, preferably less than 15% by mass, with particular preference less than 10% by mass and especially less than 5% by mass of compounds which are not fatty acid esters.

Preferably, the inventive mixture comprising epoxidized fatty acid esters contains less than 10% by mass, preferably less than 8% by mass, more preferably less than 6% by mass and especially less than 4% by mass of non-epoxidized compounds containing fatty acid chains and having no functional groups including multiple bonds in the fatty acid chain, based on the entirety of all the epoxidized and non-epoxidized compounds containing fatty acid chains.

The present invention preferably provides a mixture comprising epoxidized fatty acid esters
in which the proportion by mass of compounds containing fatty acid chains and having no functional groups including multiple bonds in the fatty acid chain has been reduced by at least 20%, preferably by at least 40%, with preference by at least 60% and especially by at least 80% compared to the mixture of the compounds containing fatty acid chains from which the mixture comprising epoxidized fatty acid esters has been prepared,
in which the alkoxide residues of the fatty acid esters are selected from alkoxide residues containing 1 to 20, preferably 2 to 15 and especially 4 to 11 carbon atoms and not having any further functional groups including multiple bonds (aside from the alkoxide function), where, in the case that the mixture comprising epoxidized fatty acid esters comprises epoxidized fatty acid esters of iso-nonanol, the proportion of derivatives of the alkoxide residue of n-nonanol in the mixture comprising epoxidized iso-nonyl fatty acid esters is less than 2 mol %, based on the entirety of all the epoxidized iso-nonyl fatty acid esters, and/or the proportion of iso-butene which has been converted in the synthesis of the precursor of the iso-nonanol used in the preparation of the iso-nonyl fatty acid esters, based on the entirety of the butene used for preparation of the iso-nonanol, is greater than 0.5 mol %, and
in which preferably less than 20% by mass, preferably less than 15% by mass, with particular preference less than 10% by mass and especially less than 5% by mass of compounds which are not fatty acid esters is present.

The present invention additionally provides a process for preparing a mixture comprising epoxidized fatty acid esters, comprising the following process steps:
reducing the proportion of the fatty acids or fatty acid esters which do not have any functional groups including multiple bonds in the fatty acid chain prior to the epoxidation of the fatty acids/fatty acid esters
OR
reducing the proportion of the fatty acids or fatty acid esters which do not have any functional groups including multiple bonds in the fatty acid chain after the epoxidation of the fatty acids or fatty acid esters,
an esterification in the case of epoxidized fatty acids and
optionally a transesterification in the case of epoxidized fatty acid esters.

The present invention accordingly provides a process for preparing a mixture comprising epoxidized fatty acid esters, comprising the following process steps:
reducing the proportion of saturated fatty acids or saturated fatty acid esters prior to the epoxidation of the fatty acids/fatty acid esters
OR
reducing the proportion of non-epoxidized fatty acids or non-epoxidized fatty acid esters after the epoxidation of the fatty acids or fatty acid esters,
an esterification in the case of epoxidized fatty acids and
optionally a transesterification in the case of epoxidized fatty acid esters.

By means of this process and the processes described hereinafter, it is possible to prepare the above-described mixtures comprising epoxidized fatty acid esters.

In one embodiment, the process according to the invention is characterized by the following steps:
a) obtaining a fatty acid mixture or fatty acid ester mixture, preferably from a vegetable oil or a mixture of at least two vegetable oils,
b) reducing the proportion of the fatty acids or the fatty acid esters which do not have any functional groups including multiple bonds in the fatty acid chain in the mixture from step a),
c) epoxidizing the mixture from step b),
d) esterifying or optionally transesterifying the mixture from step c).

Preference is given to a process characterized by the following steps:
a) obtaining a fatty acid mixture or fatty acid ester mixture, preferably from a vegetable oil or a mixture of at least two vegetable oils,
b) reducing the proportion of the saturated fatty acids or of the saturated fatty acid esters in the mixture from step a),
c) epoxidizing the mixture from step b),
d) esterifying or optionally transesterifying the mixture from step c).

The process according to the invention may comprise the following steps:
a) obtaining a fatty acid mixture, preferably from a vegetable oil or a mixture of at least two vegetable oils,
b) reducing the proportion of the saturated fatty acids in the fatty acid mixture from step a), preferably by means of crystallization,
c) epoxidizing the fatty acid mixture from step b),
d) esterifying the mixture comprising epoxidized fatty acids from step c).

Alternatively, the process according to the invention may comprise the following steps:
a) obtaining a fatty acid ester mixture, preferably from a vegetable oil or a mixture of at least two vegetable oils,
b) reducing the proportion of the saturated fatty acid esters in the fatty acid ester mixture from step a), preferably by means of crystallization,
c) epoxidizing the fatty acid ester mixture from step b),
d) optionally transesterifying the mixture comprising epoxidized fatty acid esters from step c).

In this process, there may additionally be a transesterification between steps b) and c).

The reduction in the proportion of saturated fatty acids or saturated fatty acid esters is preferably conducted by means of crystallization, especially by means of crystallization after mixing of the fatty acids or fatty acid esters with urea and alcohol, for example ethanol or methanol.

In another embodiment, the process according to the invention comprises the following steps:
a) obtaining a fatty acid mixture or fatty acid ester mixture, preferably from a vegetable oil or a mixture of at least two vegetable oils,
b) epoxidizing the mixture from step a),
c) reducing the proportion of the fatty acids or the fatty acid esters which do not have any functional groups including multiple bonds in the fatty acid chain in the mixture from step b),
d) esterifying or optionally transesterifying the mixture from step c).

Preference is given to a process comprising the following steps:
a) obtaining a fatty acid mixture or fatty acid ester mixture, preferably from a vegetable oil or a mixture of at least two vegetable oils,
b) epoxidizing the mixture from step a),
c) reducing the proportion of the non-epoxidized fatty acids or of the non-epoxidized fatty acid esters in the mixture from step b),
d) esterifying or optionally transesterifying the mixture from step c).

The process according to the invention may comprise the following steps:
a) obtaining a fatty acid ester mixture, preferably from a vegetable oil or a mixture of at least two vegetable oils,
b) epoxidizing the fatty acid ester mixture from step a),
c) reducing the proportion of the non-epoxidized fatty acid esters in the mixture comprising epoxidized fatty acid esters from step b), preferably by means of distillation,
d) optionally transesterifying the mixture comprising epoxidized fatty acid esters from step c).

Alternatively, the process according to the invention may comprise the following steps:
a) obtaining a fatty acid mixture, preferably from a vegetable oil or a mixture of at least two vegetable oils,
b) epoxidizing the fatty acid mixture from step a),
c) reducing the proportion of the non-epoxidized fatty acids in the mixture comprising epoxidized fatty acids from step b), preferably by means of distillation,
d) esterifying the mixture comprising epoxidized fatty acids from step c).

In the inventive process, additional steps such as purification or the combination of two batches for a common subsequent process step or the division of a batch for separate subsequent process steps may be conducted between step a) and step b), between step b) and step c) and/or between step c) and step d).

Preferably, in the inventive process, the proportion by mass of the compounds containing fatty acid chains and having no functional groups including multiple bonds in the fatty acid chain is reduced by at least 20%, preferably by at least 40%, with preference by at least 60% and especially by at least 80% compared to the mixture of the compounds containing fatty acid chains from which the mixture comprising epoxidized fatty acid esters has been prepared.

The present invention preferably provides a process for preparing a mixture comprising epoxidized fatty acid esters
in which the proportion by mass of the compounds containing fatty acid chains and having no functional groups including multiple bonds in the fatty acid chain is reduced by at least 20%, preferably by at least 40%, with preference by at least 60% and especially by at least 80% compared to the mixture of the compounds containing fatty acid chains from which the mixture comprising epoxidized fatty acid esters has been prepared,
in which the alkoxide residues of the fatty acid esters are selected from alkoxide residues containing 1 to 20, preferably 2 to 15 and especially 4 to 11 carbon atoms and not having any further functional groups including multiple bonds (aside from the alkoxide function), where, in the case that the mixture comprising epoxidized fatty acid esters comprises epoxidized fatty acid esters of iso-nonanol, the proportion of derivatives of the alkoxide residue of n-nonanol in the mixture comprising epoxidized iso-nonyl fatty acid esters is less than 2 mol %, based on the entirety of all the epoxidized iso-nonyl fatty acid esters, and/or the proportion of iso-butene which has been converted in the synthesis of the precursor of the iso-nonanol used in the preparation of the iso-nonyl fatty acid esters, based on the entirety of the butene used for preparation of the iso-nonanol, is greater than 0.5 mol %, and
in which, preferably, a mixture in which less than 20% by mass, preferably less than 15% by mass, with particular preference less than 10% by mass and especially less than 5% by mass of compounds which are not fatty acid esters are present is prepared,
comprising the following process steps:
   reducing the proportion of the fatty acids or fatty acid esters which do not have any functional groups including multiple bonds in the fatty acid chain prior to the epoxidation of the fatty acids/fatty acid esters
   OR
   reducing the proportion of the fatty acids or fatty acid esters which do not have any functional groups including multiple bonds in the fatty acid chain after the epoxidation of the fatty acids or fatty acid esters,
an esterification in the case of epoxidized fatty acids and
optionally a transesterification in the case of epoxidized fatty acid esters.

Preference is given to a process for preparing a mixture comprising epoxidized fatty acid esters,
in which the proportion by mass of the compounds containing fatty acid chains and having no functional groups including multiple bonds in the fatty acid chain is reduced by at least 20%, preferably by at least 40%, with preference by at least 60% and especially by at least 80% compared to the mixture of the compounds containing fatty acid chains from which the mixture comprising epoxidized fatty acid esters has been prepared,
in which the alkoxide residues of the fatty acid esters are selected from alkoxide residues containing 1 to 20, preferably 2 to 15 and especially 4 to 11 carbon atoms and not having any further functional groups including multiple bonds (aside from the alkoxide function), where, in the case that the mixture comprising epoxidized fatty acid esters comprises epoxidized fatty acid esters of iso-nonanol, the proportion of derivatives of the alkoxide residue of n-nonanol in the mixture comprising epoxidized iso-nonyl fatty acid esters is less than 2 mol %, based on the entirety of all the epoxidized iso-nonyl fatty acid esters, and/or the proportion of iso-butene which has been converted in the synthesis of the precursor of the iso-nonanol used in the preparation of the iso-nonyl fatty acid esters, based on the entirety of the butene used for preparation of the iso-nonanol, is greater than 0.5 mol %, and
in which, preferably, a mixture in which less than 20% by mass, preferably less than 15% by mass, with particular preference less than 10% by mass and especially less than 5% by mass of compounds which are not fatty acid esters are present is prepared,
comprising the following steps:
a) obtaining a fatty acid ester mixture, preferably from a vegetable oil or a mixture of at least two vegetable oils,
b) epoxidizing the fatty acid ester mixture from step a),
c) reducing the proportion of the non-epoxidized fatty acid esters in the mixture comprising epoxidized fatty acid esters from step b), preferably by means of distillation,
d) optionally transesterifying the mixture comprising epoxidized fatty acid esters from step c).

The present invention additionally provides a mixture comprising epoxidized fatty acid esters which has been prepared by one of the processes described.

The present invention provides a mixture comprising epoxidized fatty acid esters prepared by
reducing the proportion of the fatty acids or fatty acid esters which do not have any functional groups including multiple bonds in the fatty acid chain prior to the epoxidation of the fatty acids or fatty acid esters
OR
reducing the proportion of the fatty acids or fatty acid esters which do not have any functional groups including multiple bonds in the fatty acid chain after the epoxidation of the fatty acids or fatty acid esters,
an esterification in the case of epoxidized fatty acids and
optionally a transesterification in the case of epoxidized fatty acid esters.

Preference is given to a mixture comprising epoxidized fatty acid esters prepared by
reducing the proportion of saturated fatty acids or saturated fatty acid esters prior to the epoxidation of the fatty acids or fatty acid esters
OR
reducing the proportion of non-epoxidized fatty acids or non-epoxidized fatty acid esters after the epoxidation of the fatty acids or fatty acid esters,
an esterification in the case of epoxidized fatty acids and
optionally a transesterification in the case of epoxidized fatty acid esters.

Preferably, the mixture according to the invention was prepared by
a) obtaining a fatty acid mixture or fatty acid ester mixture, preferably from a vegetable oil or a mixture of at least two vegetable oils,
b) reducing the proportion of the saturated fatty acids or of the saturated fatty acid esters in the mixture from step a), preferably by means of crystallization, for example after addition of urea and alcohol,
c) epoxidizing the mixture from step b),
d) esterifying or optionally transesterifying the mixture from step c).

If, in this process, a fatty acid ester mixture is used in step a), a transesterification optionally takes place between steps b) and c).

Preference is likewise given to the preparation of the mixture according to the invention by
a) obtaining a fatty acid mixture or fatty acid ester mixture, preferably from a vegetable oil or a mixture of at least two vegetable oils,
b) epoxidizing the mixture from step a),
c) reducing the proportion of the non-epoxidized fatty acids or of the non-epoxidized fatty acid esters in the mixture from step b), preferably by means of distillation,
d) esterifying or optionally transesterifying the mixture from step c).

The present invention preferably provides a mixture comprising epoxidized fatty acid esters
in which the proportion by mass of compounds containing fatty acid chains and having no functional groups including multiple bonds in the fatty acid chain has been reduced by at least 20%, preferably by at least 40%, with preference by at least 60% and especially by at least 80% compared to the mixture of the compounds containing fatty acid chains from which the mixture comprising epoxidized fatty acid esters has been prepared,
in which the alkoxide residues of the fatty acid esters are selected from alkoxide residues containing 1 to 20, preferably 2 to 15 and especially 4 to 11 carbon atoms and not having any further functional groups including multiple bonds (aside from the alkoxide function), where, in the case that the mixture comprising epoxidized fatty acid esters comprises epoxidized fatty acid esters of iso-nonanol, the proportion of derivatives of the alkoxide residue of n-nonanol in the mixture comprising epoxidized iso-nonyl fatty acid esters is less than 2 mol %, based on the entirety of all the epoxidized iso-nonyl fatty acid esters, and/or the proportion of iso-butene which has been converted in the synthesis of the precursor of the iso-nonanol used in the preparation of the iso-nonyl fatty acid esters, based on the entirety of the butene used for preparation of the iso-nonanol, is greater than 0.5 mol %,
in which preferably less than 20% by mass, preferably less than 15% by mass, with particular preference less than 10% by mass and especially less than 5% by mass of compounds which are not fatty acid esters is present,
and which has been prepared by
   reducing the proportion of the fatty acids or fatty acid esters which do not have any functional groups including multiple bonds in the fatty acid chain prior to the epoxidation of the fatty acids or fatty acid esters
   OR
   reducing the proportion of the fatty acids or fatty acid esters which do not have any functional groups including multiple bonds in the fatty acid chain after the epoxidation of the fatty acids or fatty acid esters,
   an esterification in the case of epoxidized fatty acids and
   optionally a transesterification in the case of epoxidized fatty acid esters.

Preference is given to a mixture which comprises epoxidized fatty acid esters
in which the proportion by mass of compounds containing fatty acid chains and having no functional groups including multiple bonds in the fatty acid chain has been reduced by at least 20%, preferably by at least 40%, with preference by at least 60% and especially by at least 80% compared to the mixture of the compounds containing fatty acid chains from which the mixture comprising epoxidized fatty acid esters has been prepared,
in which the alkoxide residues of the fatty acid esters are selected from alkoxide residues containing 1 to 20, preferably 2 to 15 and especially 4 to 11 carbon atoms and not having any further functional groups including multiple bonds (aside from the alkoxide function), where, in the case that the mixture comprising epoxidized fatty acid esters comprises epoxidized fatty acid esters of iso-nonanol, the proportion of derivatives of the alkoxide residue of n-nonanol in the mixture comprising epoxidized iso-nonyl fatty acid esters is less than 2 mol %, based on the entirety of all the epoxidized iso-nonyl fatty acid esters, and/or the proportion of iso-butene which has been converted in the synthesis of the precursor of the iso-nonanol used in the preparation of the iso-nonyl fatty acid esters, based on the entirety of the butene used for preparation of the iso-nonanol, is greater than 0.5 mol %,
in which preferably less than 20% by mass, preferably less than 15% by mass, with particular preference less than 10% by mass and especially less than 5% by mass of compounds which are not fatty acid esters is present,
and which has been prepared by
a) obtaining a fatty acid ester mixture, preferably from a vegetable oil or a mixture of at least two vegetable oils,
b) epoxidizing the fatty acid ester mixture from step a),
c) reducing the proportion of the non-epoxidized fatty acid esters in the mixture comprising epoxidized fatty acid esters from step b), preferably by means of distillation,
d) optionally transesterifying the mixture comprising epoxidized fatty acid esters from step c).

The present invention additionally provides for the use of a mixture according to the invention as plasticizer for polymers.

Suitable polymers are preferably selected from the group which is formed by polyvinyl chloride (PVC), homo- or copolymers based on ethylene, propylene, butadiene, vinyl acetate, glycidyl acrylate, glycidyl methacrylate, ethyl acrylate, butyl acrylate or methacrylate with alkoxy residues from branched or unbranched alcohols having one to ten carbon atom(s), acrylonitrile or cyclic olefins, polyvinylidene chloride (PVDC), polyacrylates, especially polymethylmethacrylate (PMMA), polyalkylmethacrylate (PAMA), polyureas, silylated polymers, fluoropolymers, especially polyvinylidene fluoride (PVDF), polytetrafluoroethylene (PTFE), polyvinyl acetate (PVAc), polyvinyl alcohol (PVA), polyvinyl acetals, especially polyvinyl butyral (PVB), polystyrene polymers, especially polystyrene (PS), expandable polystyrene (EPS), acrylonitrile-styrene-acrylate (ASA), styrene-acrylonitrile (SAN), acrylonitrile-butadiene-styrene (ABS), styrene-maleic anhydride copolymer (SMA), styrene-methacrylic acid copolymer, polyolefins, especially polyethylene (PE) or polypropylene (PP), thermoplastic polyolefins (TPO), polyethylene-vinyl acetate (EVA), polycarbonates, polyethylene terephthalate (PET), polybutylene terephthalate (PBT), polyoxymethylene (POM), polyamide (PA), polyethylene glycol (PEG), polyurethane (PU), thermoplastic polyurethane (TPU), polysulphides (PSu), biopolymers, especially polylactic acid (PLA), polyhydroxybutyral (PHB), polyhydroxyvaleric acid (PHV), polyesters, starch, cellulose and cellulose derivatives, especially nitrocellulose (NC), ethylcellulose (EC), cellulose acetate (CA), cellulose acetate/butyrate (CAB), rubber and silicones.

Preferred polymers are polyvinyl chloride, copolymers of vinyl chloride with vinyl acetate or with butyl acrylate, polyalkylmethacrylate (PAMA), polyvinyl butyral (PVB), polyurethane, polysulphides, polylactic acid (PLA), polyhydroxybutyral (PHB) and nitrocellulose.

The present invention further provides a composition comprising a mixture according to the invention and one or more polymers from the group formed by polyvinyl chloride, copolymers of vinyl chloride with vinyl acetate or with butyl acrylate, polyalkyl methacrylate (PAMA), polyvinyl butyral (PVB), polyurethane, polysulphides, polylactic acid (PLA), polyhydroxybutyral (PHB) and nitrocellulose.

Based on 100 parts by mass of polymer, preferred compositions contain preferably from 5 to 200, more preferably from 10 to 150, parts by mass of plasticizer.

Preference is given to the use of the mixture according to the invention as a plasticizer for polyvinyl chloride, and particular preference is accordingly given to compositions comprising the mixture according to the invention and PVC.

Preference is given to the polymer in the form of suspension, bulk, microsuspension or emulsion PVC.

Preferred compositions according to the invention comprise, as well as the mixture according to the invention, at least one further plasticizer. A particularly preferred embodiment of the composition according to the invention exists when it contains less than 5% by mass of phthalate-containing compounds and is especially phthalate-free. The further plasticizers are preferably selected from the group of the adipates, benzoates, chlorinated hydrocarbons, citrates, cyclohexanedicarboxylates, epoxidized fatty acid esters, epoxidized vegetable oils, epoxidized acetylated glycerides, furandicarboxylates, phosphates, phthalates (preferably in minimum amounts), succinates, sulphonamides, sulphonates, terephthalates, trimellitates, or oligomeric or polymeric esters based on adipic acid, succinic acid or sebacic acid. Particular preference is given to alkyl benzoates, dialkyl adipates, glycerol esters, trialkyl citrates, acylated trialkyl citrates, trialkyl trimellitates, glycol dibenzoates, dialkyl terephthalates, esters of furandicarboxylic acid, dialkanoyl esters of dianhydrohexitols (e.g. isosorbitol) and dialkyl esters of cyclohexane-1,2-, -1,3- or -1,4-dicarboxylic acid.

In one embodiment, the composition according to the invention comprises, aside from the mixture according to the invention comprising epoxidized fatty acid esters, less than 20% by mass, less than 10% by mass or no further plasticizers, with the percentages by mass are based on the total mass of the composition.

Compositions according to the invention preferably comprise, as well as the polymer or a mixture of a plurality of polymers and the mixture according to the invention, one or more additives from the group of the thermal stabilizers, fillers, pigments, blowing agents, biocides, UV stabilizers, co-stabilizers, antioxidants, viscosity regulators, lubricants and colourants.

The present invention additionally provides for the use of the mixture according to the invention for improving the gelation of a plastisol and/or for reducing the viscosity of a plastisol and/or for reducing the glass transition temperature of a composition prepared with the inventive mixture and/or for improving the thermal stability of a composition prepared with the inventive mixture.

The mixtures according to the invention or the compositions according to the invention can be used in adhesives, sealing compounds, coating compounds, coating materials, paints, plastisols, foams, synthetic leathers, floor coverings (e.g. top layer), roofing membranes, underbody protection, fabric coatings, cables or wire insulations, hoses, extrusion articles, and in films, especially for the automobile interior sector and also in wallpaper or inks.

### EXAMPLES

### Experimental

In the experimental section, the terms “saturated”, “depleted” and “non-depleted” are used as defined in the description.

### Example 1

### Preparation of the Epoxidized Fatty Acid Methyl Ester from Linseed Oil by Epoxidation of Linseed Oil Methyl Ester

The fatty acid methyl ester (588 g, about 2 mol, Mosselmann, Methyl esters of linseed oil) was initially charged in an epoxidation apparatus (jacketed reactor with integrated cooling coil, stirrer, immersed tube, dropping funnel, metering pump, thermometer, pH meter and reflux condenser on top). The apparatus was subsequently purged with 61 of N₂/h through the immersed tube for 60 minutes. During the reaction, nitrogen was passed through the reactor contents in order to ensure inertization of the gas phase. The starting material was heated up gradually to 45° C. while stirring. On attainment of the target temperature, the pH was adjusted to pH 4.5 by means of addition of sodium hydroxide solution (20% by mass in water). A total of 690 ml of sodium hydroxide solution were added over the course of the reaction. Subsequently, peracetic acid (35%, PERACLEAN 35, Evonik Industries, 3.96 mol) was added by means of the metering pump over 2 hours. Over the whole time, the pH was kept constant by dropwise addition of sodium hydroxide solution. The reaction temperature was kept at 45° C. (+/−1.5° C.) over the entire reaction time. After the start of the reaction, the heat of reaction was removed via the cooling coil (cooling in intervals). On completion of addition, reaction was continued for 22 hours.

The reaction output was transferred to a separating funnel and left to stand at room temperature for 30 minutes. The aqueous phase was discharged and discarded. The organic phase was transferred to a 2 litre reaction flask and connected to an immersed tube, stirrer and thermometer, and also to a Claisen system with vacuum connection. The reaction output was washed three times with 25% water, based on weight. This was followed by drying at 60° C. under maximum vacuum for 15 minutes and heating to 160° C. On attainment of the temperature, the flask contents were stripped with nitrogen. For this purpose, the nitrogen rate was adjusted such that the maximum vacuum pressure rises to 40 mbar. After two hours, the heating was switched off and the mixture was cooled to 90° C. while introducing nitrogen. The ester was filtered into a suction flask by means of reduced pressure through a Büchner funnel with filter paper and a pre-compacted filtercake of filtration aid (perlite type D14).

### Preparation of the “Depleted” Mixtures, NMR and GC-MS

### Example 2

### Preparation of a “Depleted” Mixture Comprising Epoxidized Fatty Acid Methyl Esters by Reducing the Proportion of the Non-Epoxidized Fatty Acid Methyl Ester in a Mixture Comprising Epoxidized Fatty Acid Methyl Esters

### Example 2-S; From Soya Oil

### Example 2-L; From Linseed Oil

5000 g of the respective epoxidized fatty acid methyl ester were distilled in a short-path evaporator of the KDL 5 type (from UIC GmbH; evaporator, distillate stream and reflux stream can be heated separately by means of thermostats) under the following conditions:

TABLE 1
experimental data for Example 2-S and Example 2-L

| | Epox. fatty | | Epox. fatty | |
|---|---|---|---|---|
| | acid methyl ester | | acid methyl ester | |
| | formed from | | formed from | |
| | soya oil | | linseed oil | |
| | (Example 2-S) | | (Example 2-L) | |
| Pressure | <10⁻³ | mbar | <10⁻³ | mbar |
| Temperature in the evaporator | 120° | C. | 116° | C. |
| Distillate temperature | 40° | C. | 27° | C. |
| Residue temperature | 40° | C. | 40° | C. |
| Wiper speed | 313 | rpm | 305 | rpm |
| Intake pump speed | 400 | rpm | 300 | rpm |
| Residue | 74% | by mass | 81% | by mass |
| (=“depleted” mixture) | | | | |
| Distillate | 26% | by mass | 19% | by mass |

Epox. fatty acid methyl ester formed from soya oil: Reflex100 (from PolyOne)
Epox. fatty acid methyl ester formed from linseed oil: from Example 1

### Example 3

### Determination of the Composition of Mixtures Comprising Epoxidized Fatty Acid Methyl Esters by Means of “on Column” High-Temperature GC-MS with FID

Analysis parameters:
Instrument: Agilent GC 7890/Agilent MSD 5975
Capillary column: 30 m DB-5HT; internal diameter 0.32 mm; film 0.1 μm
Ionization: electron impact ionization, 70 eV, and chemical ionization with ammonia as reactant gas
Carrier gas: helium
Column flow rate: 2.6 ml/min
Oven temperature: 80° C.-10° C./min-400° C. (20 min)
Detector (FID): 400° C.
Injector: cool-on-column, 80° C.-140° C./min-400° C.

Injection volume: 0.5 μl
Sample preparation: weigh in 100 mg of the sample and 20 mg of n-hexadecane together, and add 40.0 ml of n-heptane

Identification of the Fatty Acid Methyl Esters

The fatty acid methyl esters were identified by comparison of the retention times in the sample solutions and a standard solution comprising known fatty acid methyl esters in combination with a structure elucidation by means of GC-MS analysis.

Quantification of the Fatty Acid Methyl Esters

The quantitative evaluation of the fatty acid methyl esters was conducted against n-hexadecane as internal standard, employing theoretically calculated correction factors:

Content in % by mass=(peak area for fatty acid methyl ester×calculated correction factor×weight of internal standard×100%)/(peak area for internal standard×sample weight)

Calculated Correction Factors:

| | |
|---|---|
| methyl hexadecanoate | 1.21 |
| methyl octadecanoate | 1.19 |
| methyl eicosanoate | 1.17 |
| methyl epoxyoctadecanoate | 1.40 |
| methyl diepoxyoctadecanoate | 1.47 |
| methyl triepoxyoctadecanoate | 1.63 |

TABLE 2
Composition of the mixtures comprising epoxidized fatty acid
methyl esters formed from soya oil (by GC-MS), figures in
% by mass for all compounds containing fatty acid chains

| | “Non-depleted” | “Depleted” |
|---|---|---|
| | mixture S | mixture S |
| | (reactant from | (residue from |
| | Example 2-S) | Example 2-S) |
| methyl hexadecanoate | 9.53 | 0.33 |
| methyl octadecanoate | 3.71 | 1.06 |
| methyl epoxyoctadecanoate | 23.1 | 20.0 |
| methyl eicosanoate | 0.35 | 0.26 |
| methyl diepoxyoctadecanoate | 34.2 | 41.6 |
| methyl diepoxyoctadecanoate | 19.7 | 24.9 |
| methyl docosanoate | 0.32 | 0.38 |
| methyl triepoxyoctadecanoate | 2.68 | 3.73 |
| methyl triepoxyoctadecanoate | 1.76 | 2.47 |
| methyl triepoxyoctadecanoate | 1.72 | 2.41 |
| methyl triepoxyoctadecanoate | 0.79 | 1.12 |
| Sum total of “saturated” esters | 13.91 | 2.03 |

In the mixture comprising epoxidized fatty acid methyl esters based on soya oil (“depleted” mixture S according to the invention), the proportion by mass of the compounds containing fatty acid chains which do not had any functional groups including multiple bonds in the fatty acid chain was reduced by 85.4% compared to the mixture of the compounds containing fatty acid chains from which the mixture comprising epoxidized fatty acid esters was prepared (“non-depleted” mixture, reactant from Example 2-S).

TABLE 3
Composition of the mixtures comprising epoxidized fatty acid
methyl esters formed from linseed oil (by GC-MS), figures in
% by mass for all compounds containing fatty acid chains

| | “Non-depleted” | “Depleted” |
|---|---|---|
| | mixture L | mixture L |
| | (reactant from | (residue from |
| | Example 2-L) | Example 2-L) |
| methyl hexadecanoate | 4.30 | 0.20 |
| methyl octadecanoate | 3.76 | 0.79 |
| methyl epoxyoctadecanoate | 21.9 | 15.8 |
| methyl eicosanoate | 0.13 | 0.08 |
| methyl diepoxyoctadecanoate | 11.2 | 11.8 |
| methyl diepoxyoctadecanoate | 4.53 | 5.11 |
| methyl diepoxyoctadecanoate | 8.50 | 9.17 |
| methyl triepoxyoctadecanoate | 2.62 | 3.02 |
| methyl triepoxyoctadecanoate | 17.1 | 20.4 |
| methyl triepoxyoctadecanoate | 10.6 | 12.1 |
| methyl triepoxyoctadecanoate | 8.26 | 9.12 |
| methyl triepoxyoctadecanoate | 4.53 | 5.25 |
| Sum total of “saturated” esters | 8.19 | 1.07 |

In the mixture comprising epoxidized fatty acid methyl esters based on linseed oil (“depleted” mixture L according to the invention), the proportion by mass of the compounds containing fatty acid chains which do not had any functional groups including multiple bonds in the fatty acid chain was reduced by 86.9% compared to the mixture of the compounds containing fatty acid chains from which the mixture comprising epoxidized fatty acid esters was prepared (“non-depleted” mixture, reactant from Example 2-L).

No conclusion can be drawn from the mass spectra as to the position of the epoxide groups in the fatty acid chain. In the case of polyepoxidized fatty acid esters, regio- and diastereomers occur, which explains why individual compounds were mentioned more than once in Tables 2 and 3.

### Example 4

### Determination of the Double Bond Content and the Number of Epoxide Groups Per Fatty Acid Chain by Means of NMR in Mixtures Comprising Epoxidized Fatty Acid Methyl Esters

The proportion of double bonds and epoxide groups was determined by ¹H NMR spectroscopy. For the recording of the spectra, for example, 50 mg of substance were dissolved in 0.6 ml of CDCl₃ (containing 1% by mass of TMS) and transferred to an NMR tube having a diameter of 5 mm.

The NMR spectroscopic studies can in principle be conducted with any commercial NMR instrument. For the present NMR spectroscopic studies, an instrument of the Bruker Avance 500 type was used. The spectra were recorded at a temperature of 303 K with a delay of d1=5 seconds, 32 scans, a pulse length of about 9.5 μs and a sweep width of 10 000 Hz with a 5 mm BBO (broad band observer) sample head. The resonance signals were recorded against the chemical shift of tetramethylsilane (TMS=0 ppm) as internal standard. Other commercial NMR instruments give comparable results with the same operating parameters.

To determine the proportions of the individual structural elements, the corresponding signals in the NMR spectrum first had to be identified. The signals used were listed hereinafter, with their position in the spectrum and the assignment to corresponding structural elements:
The signals in the range of 4.8 to 6.4 ppm were assigned to the ¹H nuclei of the double bonds.
The signals in the range of 3.25 to 2.85 ppm were assigned to the ¹H nuclei of the epoxides.

For quantification of the proportions, reference signals of known size were required. The following signals were used:
The signals for the methylene group adjacent to the carboxyl group of the fatty acid, which resonate as a narrow single multiplet around 2.3 ppm in the spectrum.
The signals for the methylene group adjacent to the oxygen of the esterified alcohol, corresponding to the structural element —CH₂—O—, which resonate in the range of 3.9 to 4.2 ppm in the spectrum.

The quantification was effected by determining the area beneath the respective resonance signals, i.e. the area enclosed by the signal from the baseline. Commercial NMR instruments had devices for integration of the signal area. In the present NMR spectroscopy study, the integration was conducted with the aid of the “TOPSPIN” software, Version 3.1.

To calculate the proportion of double bonds, the integral value x of the double bond signals in the range of 4.8 to 6.4 ppm was divided by the integral value for the reference methylene group r.

To calculate the proportion of epoxides, the integral value y of the epoxide signals in the range of 2.85 to 3.25 ppm was divided by the integral value for the reference methylene group r.

The relative proportions of the double bond and epoxide group structural elements per fatty acid residue/fatty acid chain were obtained.

TABLE 4
Double bond contents and number of epoxide groups per fatty acid chain

| | “Non-depleted” | “Depleted” | “Non-depleted” | “Depleted” |
|---|---|---|---|---|
| | mixture S | mixture S | mixture L | mixture L |
| | (reactant from | (residue from | (reactant from | (residue from |
| | Example 2-S) | Example 2-S) | Example 2-L) | Example 2-L) |
| Double bond content | 0.01 | 0.01 | 0.13 | 0.14 |
| Number of | 1.49 | 1.76 | 1.85 | 2.11 |
| epoxide groups | | | | |

### Examples 5 to 9

Production of the mixtures according to the invention by transesterification of the “depleted” mixture S and the “depleted” mixture L (residue from Example 2-S and Example 2-L)

The residue from Example 2-S (mₑₛₜₑᵣ) or from Example 2-L (mₑₛₜₑᵣ) was initially charged together with the particular alcohol (m_{alcohol}) n-butanol, n-pentanol, 2-ethylhexanol, iso-nonanol or 2-propylheptanol in a transesterification apparatus having a reaction flask, stirrer, immersed tube, thermometer, distillation head, 20 cm Raschig ring column, vacuum divider and collecting flask, purged with 6 l of N₂/hour through the immersed tube for one hour and heated up to 45° C. On attainment of 45° C., potassium methoxide (KOMe, Evonik Industries, 32% in methanol, m_{cat}) was added as catalyst, and gentle vacuum was applied at first. Depending on the top temperature, the vacuum was maximized as far as possible without distilling off any higher polyhydric alcohol together with the methanol. The collected alcohol was weighed (collecting flask and cold trap, m_{distillate}). At the end of the reaction, there was a vacuum of p_{vacuum}. The course of the reaction was monitored by means of GC analysis. For this purpose, a sample was taken every hour, the catalyst was admixed with acetic acid and the sample was filtered through a syringe filter. The reaction had ended when the methyl ester content was <2 area % (t_{reaction}). If the methyl ester content was still more than 2 area % after t_{further addition}, another m_{further alcohol addition} of the alcohol component and m_{further cat. addition} of KOMe were fed in. After the reaction had ended, the catalyst was broken down by adding m_{HOAc} of acetic acid (HOAc), and then the organic phase was separated from the aqueous phase in each case.

The reaction output from the transesterification was transferred to a reaction flask equipped with a Claisen system including a vacuum divider, immersed tube with nitrogen connection and thermometer, and admixed with 2% activated carbon, based on the mass of reaction output. The mixture was purged with nitrogen while stirring. The mixture was heated gradually under maximum vacuum (<1 mbar) and the temperature was increased gradually according to the onset of distillation up to T_{distillation}. A low boiler mass of m_{low boilers} was removed and then discarded. The reaction mixture was cooled down to T_{filtration} and then filtered. For this purpose, the ester was filtered into a suction flask by means of reduced pressure through a Büchner funnel with filter paper and a pre-compacted filtercake of filtration aid (perlite type D14).

### Example 5

### Transesterification of the Mixture—Depleted of Non-Epoxidized Fatty Acid Methyl Esters—Comprising Epoxidized Fatty Acid Methyl Esters to the Butyl Esters

### Example 5-S: From Soya Oil; Example 5-L: From Linseed Oil

Alcohol=butanol (Sigma Aldrich, >99%)

TABLE 5
Preparation of the butyl esters of the “depleted”
mixture S and the “depleted” mixture L

| | Butyl esters of | | Butyl esters of | |
|---|---|---|---|---|
| | “depleted” | | “depleted” | |
| | mixture S | | mixture L | |
| Starting material | residue from Example | | residue from Example | |
| | 2-S | | 2-L | |
| mₑₛₜₑᵣ | 685 | g | 708 | g |
| m_{alcohol} | 233.5 | g | 233.5 | g |
| m_{cat} | 59 | g | 59 | g |
| m_{distillate} | 290.5 | g | 290.5 | g |
| m_{further alcohol addition} | 100 | g | 100 | g |
| m_{further cat. addition} | 59 | g | 59 | g |
| m_{HOAc} | 47.2 | g | 47.2 | g |
| m_{low boilers} | 14.6 | g | 14.8 | g |
| T_{distillation} | 160° | C. | 160° | C. |
| T_{filtration} | <80° | C. | <80° | C. |
| t_{further addition} | 9.5 | h | 9.5 | h |
| t_{reaction} | 17.5 | h | 17.5 | h |
| p_{vacuum} | 2 | mbar | 1 | mbar |

### Example 6

### Transesterification of the Mixture—Depleted of Non-Epoxidized Fatty Acid Methyl Esters—Comprising Epoxidized Fatty Acid Methyl Esters to the Pentyl Esters

### Example 6-S: from Soya Oil; Example 6-L: From Linseed Oil

Alcohol=n-pentanol (Sigma Aldrich, >99%)

TABLE 6
Preparation of the pentyl esters of the “depleted”
mixture S and the “depleted” mixture L

| | n-Pentyl esters of | | n-Pentyl esters of | |
|---|---|---|---|---|
| | “depleted” | | “depleted” | |
| | mixture S | | mixture L | |
| Starting material | residue from Example | | residue from Example | |
| | 2-S | | 2-L | |
| mₑₛₜₑᵣ | 685 | g | 351 | g |
| m_{alcohol} | 278 | g | 139 | g |
| m_{cat} | 59 | g | 29.5 | g |
| m_{distillate} | 233 | g | 114 | g |
| m_{further alcohol addition} | 191 | g | 114 | g |
| m_{further cat. addition} | 0 | | 0 | |
| m_{HOAc} | 23.6 | g | 11.8 | g |
| m_{low boilers} | 87 | g | 20 | g |
| T_{distillation} | 160° | C. | 160° | C. |
| T_{filtration} | <80° | C. | <80° | C. |
| t_{further addition} | 1 h, 2 h, 3 h | | 1 h, 2 h, 3 h, 4 h | |
| t_{reaction} | 4 | h | 1.5 | h |
| p_{vacuum} | <1 | mbar | <1 | mbar |

### Example 7

### Transesterification of the Mixture—Depleted of Non-Epoxidized Fatty Acid Methyl Esters—Comprising Epoxidized Fatty Acid Methyl Esters to the 2-Ethylhexyl Esters

### Example 7-S: From Soya Oil; Example 7-L: From Linseed Oil

Alcohol=2-ethylhexanol (Sigma Aldrich, >99%)

TABLE 7
Preparation of the 2-ethylhexyl esters of the “depleted”
mixture S and the “depleted” mixture L

| | 2-Ethylhexyl esters of | | 2-Ethylhexyl esters of | |
|---|---|---|---|---|
| | “depleted” | | “depleted” | |
| | mixture S | | mixture L | |
| Starting material | residue from Example | | residue from Example | |
| | 2-S | | 2-L | |
| mₑₛₜₑᵣ | 685 | g | 701 | g |
| m_{alcohol} | 410 | g | 410 | g |
| m_{cat} | 59 | g | 59 | g |
| m_{distillate} | 104 | g | 97 | g |
| m_{further alcohol addition} | 0 | | 97 | g |
| m_{further cat. addition} | 0 | | 0 | |
| m_{HOAc} | 23.6 | g | 23.6 | g |
| m_{low boilers} | 158 | g | 282 | g |
| T_{distillation} | 160° | C. | 160° | C. |
| T_{filtration} | <80° | C. | <80° | C. |
| t_{further addition} | no further addition | | 1 h, 2.5 h | |
| t_{reaction} | 4 | h | 4.5 | h |
| p_{vacuum} | 3 | mbar | 6 | mbar |

### Example 8

### Transesterification of the Mixture—Depleted of Non-Epoxidized Fatty Acid Methyl Esters—Comprising Epoxidized Fatty Acid Methyl Esters to the Iso-Nonyl Esters

### Example 8-S: from Soya Oil; Example 8-L: From Linseed Oil

Alcohol=iso-nonanol (Evonik Industries, >99%)

TABLE 8
Preparation of the iso-nonyl esters of the “depleted”
mixture S and the “depleted” mixture L

| | iso-Nonyl esters of | | iso-Nonyl esters of | |
|---|---|---|---|---|
| | “depleted” | | “depleted” | |
| | mixture S | | mixture L | |
| Starting material | residue from Example | | residue from Example | |
| | 2-S | | 2-L | |
| mₑₛₜₑᵣ | 685 | g | 701 | g |
| m_{alcohol} | 454 | g | 454 | g |
| m_{cat} | 59 | g | 59 | g |
| m_{distillate} | 108 | g | 113 | g |
| m_{further alcohol addition} | 106 | g | 113 | g |
| m_{further cat. addition} | 0 | | 0 | |
| m_{HOAc} | 23.6 | g | 23.6 | g |
| m_{low boilers} | 256 | g | 241 | g |
| T_{distillation} | 160° | C. | 160° | C. |
| T_{filtration} | <80° | C. | <80° | C. |
| t_{further addition} | 1 h, 2 h | | 1 h, 2 h | |
| t_{reaction} | 5.75 | h | 7 | h |
| p_{vacuum} | 2 | mbar | <1 | mbar |

### Example 9

### Transesterification of the Mixture—Depleted of Non-Epoxidized Fatty Acid Methyl Esters—Comprising Epoxidized Fatty Acid Methyl Esters to the 2-Propylheptyl Esters

### Example 9-S: From Soya Oil; Example 9-L: From Linseed Oil

Alcohol=2-propylheptanol (Evonik Industries, >99%)

TABLE 9
Preparation of the 2-propylheptyl esters of the “depleted”
mixture S and the “depleted” mixture L

| | 2-Propylheptyl esters of | | 2-Propylheptyl esters of | |
|---|---|---|---|---|
| | “depleted” | | “depleted” | |
| | mixture S | | mixture L | |
| Starting material | residue from Example | | residue from Example | |
| | 2-S | | 2-L | |
| mₑₛₜₑᵣ | 685 | g | 701 | g |
| m_{alcohol} | 454 | g | 498 | g |
| m_{cat} | 59 | g | 59 | g |
| m_{distillate} | 108 | g | 142 | g |
| m_{further alcohol addition} | 108 | g | 0 | |
| m_{further cat. addition} | 0 | | 0 | |
| m_{HOAc} | 23.6 | g | 23.6 | g |
| m_{low boilers} | 235 | g | 34 | g |
| T_{distillation} | 160° | C. | 160° | C. |
| T_{filtration} | 80° | C. | 80° | C. |
| t_{further addition} | 1 h, 2 h | | no further addition | |
| t_{reaction} | 3.25 | h | 7 | h |
| p_{vacuum} | 2 | mbar | 6 | mbar |

### Examples 10 to 13

### Production of the “Non-Depleted” Mixtures (Comparative Substances) by Esterification of the Epoxidized, “Non-Depleted” Fatty Acid Methyl Esters

The “non-depleted” mixtures were prepared by transesterification analogously to the method described in Examples 5 to 9, except using the respective epoxidized fatty acid methyl ester formed from soya oil (reactant from Example 2-S) or from linseed oil (reactant from Example 2-L) rather than the residue from Example 2-S or from Example 2-L.

Epox. fatty acid methyl ester formed from soya oil: reactant from Example 2-S, Reflex100 (from PolyOne)

Epox. fatty acid methyl ester formed from linseed oil: from Example 1, reactant from Example 2-L

### Example 10

### Transesterification of the “Non-Depleted” Mixture to the Butyl Esters

### Example 10-S: from Soya Oil; Example 10-L: From Linseed Oil

Alcohol=butanol (Sigma Aldrich, >99%)

TABLE 10
Preparation of the butyl esters of the “non-depleted”
mixture S and the “non-depleted” mixture L

| | Butyl esters of | | Butyl esters of | |
|---|---|---|---|---|
| | “non-depleted” | | “non-depleted” | |
| | mixture S | | mixture L | |
| Starting material | reactant from Example | | reactant from Example | |
| | 2-S | | 2-L | |
| mₑₛₜₑᵣ | 685 | g | 685 | g |
| m_{alcohol} | 233.5 | g | 233.5 | g |
| m_{cat} | 59 | g | 59 | g |
| m_{distillate} | 158 | g | 90 | g |
| m_{further alcohol addition} | 0 | | 0 | |
| m_{further cat. addition} | 0 | | 0 | |
| m_{HOAc} | 23.6 | g | 23.6 | g |
| m_{low boilers} | 14.3 | g | 14.3 | g |
| T_{distillation} | 160° | C. | 160° | C. |
| T_{filtration} | <80° | C. | <80° | C. |
| t_{further addition} | no further addition | | no further addition | |
| t_{reaction} | 9.5 | h | 8 | h |
| p_{vacuum} | <1 | mbar | 26 | mbar |

### Example 11

### Transesterification of the “Non-Depleted” Mixture to the 2-Ethylhexyl Esters

### Example 11-S: From Soya Oil; Example 11-L: From Linseed Oil

Alcohol=2-ethylhexanol (Sigma Aldrich, >99%)

TABLE 11
Preparation of the 2-ethylhexyl esters of
the “non-depleted” mixture L

| | 2-Ethylhexanol esters of | |
|---|---|---|
| | “non-depleted” | |
| | mixture L | |
| Starting material | reactant from Example | |
| | 2-L | |
| mₑₛₜₑᵣ | 996 | g |
| m_{alcohol} | 396 | g |
| m_{cat} | 84 | g |
| m_{distillate} | 100 | g |
| m_{further alcohol addition} | 100 | g |
| m_{further cat. addition} | 0 | |
| m_{HOAc} | 34 | g |
| m_{low boilers} | 219 | g |
| T_{distillation} | 160° | C. |
| T_{filtration} | <80° | C. |
| t_{further addition} | 1 h, 2 h | |
| t_{reaction} | 4 | h |
| p_{vacuum} | 8 | mbar |

### Example 12

### Transesterification of the “Non-Depleted” Mixture to the Iso-Nonyl Esters

### Example 12-S: From Soya Oil; Example 12-L: From Linseed Oil

Alcohol=iso-nonanol (Evonik Industries, >99%)

TABLE 12
Preparation of the iso-nonyl esters of the “non-depleted” mixture S and
the “non-depleted” mixture L

| | | | iso-Nonyl esters of | |
|---|---|---|---|---|
| | iso-Nonyl esters of | | “non-depleted” | |
| | “non-depleted” mixture S | | mixture L | |
| Starting material | reactant from Example | | reactant from Example | |
| | 2-S | | 2-L | |
| mₑₛₜₑᵣ | 685 | g | 685 | g |
| m_{alcohol} | 454 | g | 454 | g |
| m_{cat} | 59 | g | 59 | g |
| m_{distillate} | 117 | g | 102 | g |
| m_{further alcohol addition} | 0 | | 0 | |
| m_{further cat. addition} | 0 | | 0 | |
| m_{HOAc} | 23.6 | g | 23.6 | g |
| m_{low boilers} | 149 | g | 154 | g |
| T_{distillation} | 160° | C. | 160° | C. |
| T_{filtration} | 80° | C. | 80° | C. |
| t_{further addition} | no further addition | | no further addition | |
| t_{reaction} | 6 | h | 6 | h |
| p_{vacuum} | 3 | mbar | <1 | mbar |

### Example 13

### Transesterification of the “Non-Depleted” Mixture to the 2-Propylheptyl Esters

### Example 13-S: From Soya Oil; Example 13-L: From Linseed Oil

Alcohol=2-propylheptanol (Evonik Industries, >99%)

TABLE 13
Preparation of the 2-propylheptyl esters of the “non-depleted”
mixture S and the “non-depleted” mixture L

| | | | 2-Propylheptyl esters of | |
|---|---|---|---|---|
| | 2-Propylheptyl esters of | | “non-depleted” | |
| | “non-depleted” mixture S | | mixture L | |
| Starting material | residue from Example | | residue from Example | |
| | 2-S | | 2-L | |
| mₑₛₜₑᵣ | 685 | g | 701 | g |
| m_{alcohol} | 498 | g | 498 | g |
| m_{cat} | 59 | g | 59 | g |
| m_{distillate} | 115 | g | 118 | g |
| m_{further alcohol addition} | 115 | g | 118 | g |
| m_{further cat. addition} | 0 | | 0 | |
| m_{HOAc} | 23.6 | g | 23.6 | g |
| m_{low boilers} | 275 | g | 282 | g |
| T_{distillation} | 160° | C. | 160° | C. |
| T_{filtration} | 80° | C. | 80° | C. |
| t_{further addition} | 1 h, 2 h | | 1 h, 2.5 h | |
| t_{reaction} | 3.25 | h | 3.5 | h |
| p_{vacuum} | 2 | mbar | 4 | mbar |

### Example 14

### Transesterification of Epoxidized Soya Bean Oil (Example 14-S) or of Epoxidized Linseed Oil (Example 14-L) to the n-Pentyl Esters

In a transesterification apparatus having a reaction flask, stirrer, immersed tube, thermometer, distillation head, 20 cm Raschig ring column, vacuum divider and collecting flask, potassium methoxide (Evonik Industries AG, 32% in methanol, m_{cat}) was mixed with half the n-pentanol (m_{alcohol}/2). Under maximum vacuum (p_{vacuum}), the mixture was heated to 45° C. The distillate obtained, without reflux, was removed completely (m_{distillate}). After the reactor had been flooded with nitrogen, the second half of the alcohol was then rapidly added dropwise at constant bottom temperature. After one hour, the collecting flask and cold trap were weighed, and the distillate obtained was replaced by additional n-pentanol (m_{further alcohol addition}). This operation was repeated after the second hour of reaction time (value was included m_{further alcohol addition}). It was assumed that the potassium methoxide had been fully converted to the corresponding potassium alkoxide after two hours of reaction time. Epoxidized soya bean oil or epoxidized linseed oil (mₑₛₜₑᵣ) was rapidly added dropwise at 45° C. to the flask comprising catalyst and alcohol, and then the bottom temperature was kept constant. The course of the reaction was monitored by means of HT-GC analysis. For this purpose, a sample was taken every hour, the catalyst was admixed with acetic acid and the sample was filtered through a syringe filter. To determine the mono-, di- and triglycerides by means of HT-GC, the sample had to be silylated. As soon as the remaining residue of mono-, di- and triglycerides was less than 2 area % (t_{reaction}), the reaction was stopped by adding acetic acid (m_{HOAc}).

The contents of the flask were introduced into a separating funnel and left to stand. The glycerol (lower phase) was discharged. The upper phase was washed twice with water at 80° C. while sparging with nitrogen (30% water based on the contents of the flask).

An acid number of the organic phase was determined, and then neutralization was effected with 1.1 times the stoichiometric amount (for the particular acid number) of 10% sodium hydroxide solution. The product was then washed again with water at 80° C. while sparging with nitrogen until the washing water was neutral (pH 7-8).

The organic phase was transferred to a reaction flask equipped with a Claisen system including a vacuum divider, immersed tube with nitrogen connection and thermometer, and admixed with 2% activated carbon, based on the mass of reaction output. The mixture was purged with nitrogen while stirring. The mixture was heated gradually under maximum vacuum (<1 mbar) and the temperature was increased gradually according to the onset of distillation up to T_{distillation}. A low boiler mass of m_{low boilers} was removed and then discarded. The reaction mixture was cooled down to T_{filtration} and then filtered. For this purpose, the ester was filtered into a suction flask by means of reduced pressure through a Büchner funnel with filter paper and a pre-compacted filtercake of filtration aid (perlite type D14).

TABLE 14
Preparation of the n-pentyl esters of the “non-depleted”
mixture S and the “non- depleted” mixture L

| | n-Pentyl esters of | | n-Pentyl esters of | |
|---|---|---|---|---|
| | “non-depleted” | | “non-depleted” | |
| | mixture S | | mixture L | |
| Starting material | epoxidized soya bean | | epoxidized linseed | |
| | oil | | oil | |
| mₑₛₜₑᵣ | 996 | g | 996 | g |
| m_{alcohol} | 396 | g | 396 | g |
| m_{cat} | 84 | g | 84 | g |
| m_{distillate} | 92 | g | 100 | g |
| m_{further alcohol addition} | 235 | g | 100 | g |
| m_{further cat. addition} | 0 | | 0 | |
| m_{HOAc} | 34 | g | 34 | g |
| m_{low boilers} | 92 | g | 219 | g |
| T_{distillation} | 160° | C. | 160° | C. |
| T_{filtration} | <80° | C. | <80° | C. |
| t_{further addition} | 1 h, 2 h | | 1 h, 1 h | |
| t_{reaction} | 4 | h | 4 | h |
| p_{vacuum} | 11 | mbar | 8 | mbar |

Epoxidized soya bean oil: Epoxol D65, Avokal GmbH
Epoxidized linseed oil: Merginat ELO, Hobum Chemicals

Subsequently, distillation was effected by means of a short-path evaporator of the KDL 5 type (from UIC GmbH; evaporator, distillate stream and reflux stream can be treated separately by means of thermostats) under the conditions which follow.

TABLE 15
Distillation conditions in the preparation of the
n-pentyl esters of the “non-depleted” mixture
S and the “non-depleted” mixture L

| | n-Pentyl esters of | | n-Pentyl esters of | |
|---|---|---|---|---|
| | “non-depleted” | | “non-depleted” | |
| | mixture S | | mixture L | |
| Pressure | <10⁻³ | mbar | <10⁻³ | mbar |
| Temperature in the evaporator | 180° | C. | 180° | C. |
| Distillate temperature | 25° | C. | 25° | C. |
| Residue temperature | 80° | C. | 80° | C. |
| Wiper speed | 300 | rpm | 300 | rpm |
| Intake pump speed | 400 | rpm | 400 | rpm |
| Residue | 12% | by mass | 17% | by mass |
| Distillate | 88% | by mass | 83% | by mass |

### Comparison of the Properties of “Depleted” and “Non-Depleted” Mixtures

It was found that the “depleted” mixtures consistently had better compatibility with PVC than “non-depleted” mixtures which differ from the “depleted” mixtures merely in the proportion of the compounds containing fatty acid chains, which were referred to as “saturated” in the context of the present text.

The compatibility of the plasticizers in the PVC films examined was conducted by the “Loop test” (ASTM D 3291). In the case of the “non-depleted” mixtures of epoxidized 2-propylheptyl esters of linseed oil or soya oil, distinct sweating phenomena occurred. Therefore, use in typical flexible PVC applications was impossible. Since “non-depleted” mixtures of epoxidized 2-propylheptyl esters of linseed oil or soya oil were not compatible with PVC, the further properties of the 2-propylheptyl ester mixtures were not determined in the tests which follow. The inventive “depleted” mixture of epoxidized 2-propylheptyl esters, in contrast, was compatible with PVC, and so the properties thereof were likewise determined.

In the examples which follow, the following mixtures comprising epoxidized fatty acid esters were examined:

TABLE 16
Mixtures based on soya oil examined

| | “Non-depleted” | “Depleted” |
|---|---|---|
| | mixture S | mixture S |
| Methyl esters | Reflex 100 (from PolyOne) | from Example 2-S |
| Butyl esters | from Example 10-S | from Example 5-S |
| n-Pentyl esters | from Example 14-S | from Example 6-S |
| 2-Ethylhexyl esters | PLS Green 8 (Petrom) | from Example 7-S |
| iso-Nonyl esters | from Example 12-S | from Example 8-S |
| 2-Propylheptyl esters | from Example 13-S | from Example 9-S |

TABLE 17
Mixtures based on linseed oil examined

| | “Non-depleted” | “Depleted” |
|---|---|---|
| | mixture L | mixture L |
| Methyl esters | from Example 1 | from Example 2-L |
| Butyl esters | from Example 10-L | from Example 5-L |
| n-Pentyl esters | from Example 14-L | from Example 6-L |
| 2-Ethylhexyl esters | from Example 11-L | from Example 7-L |
| iso-Nonyl esters | from Example 12-L | from Example 8-L |
| 2-Propylheptyl esters | from Example 13-L | from Example 9-L |

### Example 15

### Volatility of the “Depleted” and the “Non-Depleted” Mixtures

### Pure Plasticizers

The volatility of plasticizers was a central property for many polymer applications. High volatilities lead to increased environmental exposure and, in the case of long product lifetimes, to worsened mechanical properties as a result of reduced plasticizer contents in the polymer. Volatile plasticizers, if they were used at all, were therefore frequently added only in small proportions to other plasticizer systems. Of particular significance was the volatility, for example, in interior applications (wallpaper, automobiles) or, because of guidelines and standards, in cables or food and drink packaging.

The volatility of the pure plasticizers was determined using the Mettler Toledo HB 43-S halogen drier. An empty, clean aluminium pan was placed on the weighing pan before the measurement. The aluminium pan was then tared with a mat and about five grams of plasticizer were pipetted onto the mat and weighed accurately.

The measurement was started by closing the heating module, and the sample was heated from room temperature to 200° C. at the maximum heating rate (default setting), and the corresponding loss of mass through evaporation was determined automatically by weighing every 30 seconds. After 10 min, the measurement was ended automatically by the instrument.

A duplicate determination was conducted for each sample.

TABLE 18
Volatility of the mixtures based on soya oil in % by mass

| | “Non-depleted” | “Depleted” |
|---|---|---|
| | mixture S | mixture S |
| Methyl esters | 21.2 | 12.8 |
| Butyl esters | 9.6 | 5.5 |
| n-Pentyl esters | 8.8 | 4.9 |
| 2-Ethylhexyl esters | 5.4 | 3.3 |
| iso-Nonyl esters | 3.4 | 2.4 |
| 2-Propylheptyl esters | — | 2.0 |

TABLE 19
Volatility of the mixtures based on linseed oil in % by mass

| | “Non-depleted” | “Depleted” |
|---|---|---|
| | mixture L | mixture L |
| Methyl esters | 16.5 | 10.3 |
| Butyl esters | 9.9 | 4.9 |
| n-Pentyl esters | 7.4 | 3.6 |
| 2-Ethylhexyl esters | 2.2 | 2.1 |
| iso-Nonyl esters | 2.2 | 1.6 |

The inventive “depleted” mixtures comprising epoxidized fatty acid esters consistently show a lower volatility than the “non-depleted” mixtures.

### Example 16

### Production of Plastisols

PVC plastisols were produced, as used, for example, for the manufacture of topcoat films for floor coverings. The figures in the plastisol formulations were each in parts by mass. The formulations of the polymer compositions were listed in Table 20.

TABLE 20
Plastisol formulation

| | phr |
|---|---|
| PVC (Vestolit B 7021 - Ultra; from Vestolit) | 100 |
| “Depleted” mixture or “non-depleted” mixture | 50 |
| Epoxidized soya bean oil as co-stabilizer (Drapex 39, from Galata) | 3 |
| Thermal stabilizer based on Ca/Zn (Mark CZ 149, from Galata) | 2 |

phr: parts per hundred parts resin

The plasticizers were equilibrated to 25° C. prior to addition. First the liquid constituents and then the pulverulent constituents were weighed out into a PE cup. The mixture was stirred manually with an ointment spatula in such a way that no unwetted powder was present any longer. The mixing beaker was then clamped into the clamping device of a dissolver stirrer. Before the stirrer was immersed into the mixture, the rotational speed was set to 1800 revolutions per minute. After switching on the stirrer, the mixture was stirred until the temperature on the digital display of the thermal sensor reached 30.0° C. This ensured that the homogenization of the plastisol was achieved for a defined energy input. Thereafter, the plastisol was immediately equilibrated to 25.0° C. in a climate-controlled cabinet for further studies.

### Example 17

### Gelation Characteristics of Plastisols Comprising “Depleted” and “Non-Depleted” Mixtures

The gelation characteristics of the plastisols were examined with a Physica MCR 101 in oscillation mode using a parallel plate analysis system (PP25), which was operated under shear stress control. An additional heating hood was connected to the system in order to achieve a homogeneous heat distribution and uniform sample temperature.

The following parameters were set:

Mode:

Temperature gradient

| Start temperature | 25° | C. |
|---|---|---|
| End temperature | 180° | C. |
| Heating/cooling rate | 5° | C./min |
| Oscillation frequency | 4-0.1 Hz logarithmic ramp | |
| Frequency cycle omega: | 10 | 1/s |
| Number of measurement points: | 63 | |
| Measurement point duration: | 0.5 | min |
| Automatic gap readjustment F: | 0N | |
| Constant measurement point duration | 0.5 | mm |
| Gap width | | |

Analysis Procedure:

The spatula was used to apply a few grams of the plastisol to be analysed, free from air bubbles, to the lower plate of the analysis system. In doing so, it was ensured that, after the analysis system had been assembled, it was possible for some plastisol to exude uniformly out of the analysis system (not more than about 6 mm in any direction). The heating hood was subsequently positioned over the sample and the analysis was started. What was called the complex viscosity of the plastisol was determined after 24 h (storage of the plastisol at 25° C. in a temperature control cabinet from Memmert) as a function of temperature.

A distinct rise in the complex viscosity was considered to be a measure of gelation. The comparative value used was therefore the temperature on attainment of a plastisol viscosity of 1000 Pa·s.

TABLE 21
Gelation of the mixtures based on soya oil after 24 h, temperature in °
C. on attainment of a plastisol viscosity of 10³ Pa · s

| | “Non-depleted” | “Depleted” |
|---|---|---|
| | mixture S | mixture S |
| Methyl esters | 60 | 56 |
| Butyl esters | 72 | 66 |
| n-Pentyl esters | 74 | 68 |
| 2-Ethylhexyl esters | 85 | 74 |
| iso-Nonyl esters | 88 | 78 |
| 2-Propylheptyl esters | — | 81 |

TABLE 22
Gelation of the mixtures based on linseed oil after 24 h, temperature in
° C. on attainment of a plastisol viscosity of 10³ Pa · s

| | “Non-depleted” | “Depleted” |
|---|---|---|
| | mixture L | mixture L |
| Methyl esters | 55 | 51 |
| Butyl esters | 63 | 61 |
| n-Pentyl esters | 66 | 65 |
| 2-Ethylhexyl esters | 78 | 71 |
| iso-Nonyl esters | 81 | 74 |
| 2-Propylheptyl esters | — | 76 |

The viscosity of 10³ Pa·s was generally achieved at lower temperatures in the case of the “depleted” mixtures of equal alcohol chain length; gelation was better. This property of the inventive “depleted” mixtures leads to advantages in the processability of corresponding plastisols, since it was thus possible either to select lower working temperatures (energy saving) or to increase the throughput by increasing the running speed of the belts on which the plastisols gelate.

### Example 18

### Efficiency of the Mixtures

### Shore A Hardness of the Flexible PVC Samples

Shore hardness was a measure of the softness of a sample. The further a standardized needle can penetrate into the sample in a particular measurement period, the lower the measured value. The plasticizer having the highest efficiency for the same amount of plasticizer gives the lowest Shore hardness value. Since, in practice, formulations were frequently adjusted or optimized to a certain Shore hardness, very efficient plasticizers can accordingly be reduced to a particular proportion in the formulation, which leads to a reduction in costs for the processor.

For determination of the Shore hardnesses, the plastisols produced as described above were poured into round brass casting moulds having a diameter of 42 mm (weight: 20.0 g). The pastes were then gelated in the moulds in an air circulation drying cabinet at 200° C. for 30 min, cooled and then removed, and stored in a climate-controlled cabinet (25° C.) for at least 24 hours prior to the measurement. The slice thickness was about 12 mm.

The hardness measurements were conducted to DIN 53 505 using a Shore A measuring instrument from Zwick-Roell; the measurement was read off after 3 seconds in each case. For each test specimen, measurements were conducted at three different positions, and the mean was determined.

TABLE 23
Shore A hardness of test specimens comprising
mixtures based on soya oil

| | “Non-depleted” | “Depleted” |
|---|---|---|
| | mixture S | mixture S |
| Methyl esters | 69 | 66 |
| Butyl esters | 73 | 70 |
| n-Pentyl esters | 74 | 71 |
| 2-Ethylhexyl esters | 80 | 74 |
| iso-Nonyl esters | 81 | 76 |
| 2-Propylheptyl esters | — | 78 |

TABLE 24
Shore A hardness of test specimens comprising
mixtures based on linseed oil

| | “Non-depleted” | “Depleted” |
|---|---|---|
| | mixture L | mixture L |
| Methyl esters | 64 | 64 |
| Butyl esters | 72 | 69 |
| n-Pentyl esters | 74 | 71 |
| 2-Ethylhexyl esters | 80 | 73 |
| iso-Nonyl esters | 81 | 74 |
| 2-Propylheptyl esters | — | 75 |

Test specimens of the “depleted” mixtures show a lower Shore A hardness and hence a better plasticizer efficiency than test specimens of the “non-depleted” mixtures. In this way, it was possible to save plasticizer, which leads to lower formulation costs.

### Example 19

### Production of Dry Blends, Rolled Sheets and Pressed Plaques

The test specimens required for the examples which follow were produced by dry mixing (dry blend production), calendering (rolling) and pressing of the following formulations:

TABLE 25
Dry blend formulation

| | phr |
|---|---|
| PVC (Solvin S271 PC; from Solvay) | 100 |
| “Depleted” mixture or “non-depleted” mixture | 67 |
| Epoxidized soya bean oil (Drapex 39; from Galata) | 3 |
| Thermal stabilizer based on Ba/Zn (Mark BZ 965; from Galata) | 2 |
| Fatty acid salts (Mark CD 41-0137; from Galata) | 0.4 |

phr: part per hundred parts resin

With dry mixtures, which were also referred to as dry blends, it was possible, for example, to produce cable and wire insulation or floors and roofing membranes.

The dry blends were produced in a Brabender planetary mixer. A thermostat (from Lauda, RC6) filled with demineralized water ensured the control of the mixing vessel temperature in the planetary mixer. A PC recorded the data transmitted by the mixer via a data cable in the “Winmix” software.

The “Winmix” software was used to set the following parameters in the Brabender planetary mixer.
Speed program: active
Profile: speed 50 rpm; hold time: 9 min; rise time: 1 min speed 100 rpm; hold time: 20 min
Kneader temperature: 88° C.
Measurement range: 2 Nm
Damping: 3

A temperature of 90° C. was set on the thermostat, and a temperature of the mixing vessel in the Brabender was controlled via a hose connection. The temperature in the mixing vessel was 88° C. after the one-hour equilibration period. Once the planetary mixer had conducted an internal calibration, the solid constituents (PVC, filler, stabilizer), which had been weighed out beforehand in four times the amount (four times the amount in g based on Table 24 in phr) into a PE cup on a balance (Mettler XS6002S), were fed to the mixing vessel via a solids funnel and the filling stub present in the Brabender mixing vessel. The program was started and the powder mixture was stirred and equilibrated in the mixing vessel for 10 minutes, before the liquid constituents, which had likewise been weighed out in four times the amount in a PE cup on the balance, were fed in via a liquid funnel and the filling stub present in the Brabender mixing vessel. The mixture was stirred in a planetary mixer for a further 20 minutes. After the program had ended, the finished dry mixture (dry blend) was removed.

These dry blends were used to produce rolled sheets. The rolled sheets were produced on a Collin W150 AP calender. The Collin calender had an automatic sample turner and its temperature was controlled by means of an additional oil thermostat (Single STO 1-6-12-DM). Control was effected by means of Collin software.

The following parameters were set in the calender:

Roll temperature [° C.]: 165

Rolling time [min]: 5.83

Five-stage program for production of the rolled sheet

| | |
|---|---|
| Stage 1: Plastification of the dry blend | (165° C./60 s/0.2 mm gap/5 |
| | rpm) |
| Stage 2: Change in roll gap | (165° C./30 s/0.5 mm gap/20 |
| | rpm) |
| Stage 3: Mixing of melt | (165° C./170 s/0.5 mm gap/20 |
| | rpm) |
| Stage 4: Rolled sheet optimization | (165° C./30 s/0.5 mm gap/25 |
| | rpm) |
| Stage 5: Rolled sheet removal | (165° C./60 s/0.5 mm gap/7 |
| | rpm) |

On attainment of the roll temperature, the roll gap was calibrated. To start the measurement, the roll gap was adjusted to 0.2 mm. 160 g of each dry blend were weighed again and introduced into the roll gap with the rollers stationary. The program was started. The rollers started with a circumferential speed of 5 rpm and a friction of 20%. After about 1 min, the plastification was complete for the most part, and the roll gap was increased to 0.5 mm. Homogenization was effected 6 times by means of the automatic turning unit in the calender. After about 6 min, the rolled sheet was removed from the roller and cooled.

The pressed plaques were produced on a Collin laboratory press. The prefabricated rolled sheets (see above) were used to produce the pressed plaques. The lateral edges of the rolled sheets were removed with the aid of a cutting machine, then the rolled sheet was cut into pieces of about 14.5×14.5 cm in size. For pressed plaques of thickness 1 mm, 2 rolled sheet pieces in each case were placed into the stainless steel pressing frame of size 15×15 cm.

The following parameters were set in the laboratory press:

Three-phase program:

Phase 1: both platens 175° C.; press platen pressure: 5 bar; phase time: 60 seconds.

Phase 2: both platens 175° C.; press platen pressure: 200 bar; phase time: 120 seconds.

Phase 3: both platens 40° C.; press platen pressure: 200 bar; phase time: 270 seconds.

The excess compression lip was removed after the press plaques had been produced.

### Example 20

### Loss of Mass in the Course of Activated Carbon Storage

3 circles of 10 cm² were punched out of each of the pressed plaques of Example 19 for each formulation to be tested. In addition, scissors were used to make radial cuts into the circles (2×5 mm cuts). The circles were equilibrated in a desiccator (filled with orange KC-Trockenperlen drying beads) for half an hour and then weighed.

Tin cans (1 l, tall shape) were punctured in the lid in order that exchange of pressure could take place. The bases of the tin cans were covered with 120 ml of activated carbon. The activated carbon used in this test (774408 from Roth) was dried beforehand in a crucible for 6 hours in a drying cabinet at 100+/−1° C., cooled briefly and then used. The first sample circle was placed onto the middle of the activated carbon. A further 120 ml of activated carbon were placed onto the sample circle. In total, the tin cans were filled with 480 ml of activated carbon and 3 sample circles in layers. The lid of the tin cans was placed onto the cans without pressure.

The filled tin cans were stored in a temperature control cabinet at 120+/−1° C. for 3 days. After the storage, the activated carbon was removed from the circles by means of an analysis brush, and the circles were stored in a desiccator for 30 minutes for cooling and then weighed.

After the weighing, the sample circles were layered again with activated carbon in the tin cans. For this purpose, it was ensured that the sample circles were again assigned to the same activated carbon and the same can. The cans were placed in the temperature control cabinet again. After a total of 7 days, the samples were then weighed again as already described.

The percentage change in mass of each sample circle was calculated, and the mean over the 3 circles for each formulation was calculated.

TABLE 26
Change in mass in the course of activated carbon
storage in % by mass (mixtures based on soya oil)

| | “Non-depleted” | | “Depleted” | |
|---|---|---|---|---|
| | mixture S | | mixture S | |
| | 3 days | 7 days | 3 days | 7 days |
| Methyl esters | −19.9 | −27.1 | −16.9 | −25.8 |
| Butyl esters | −13.8 | −19.7 | −8.2 | −13.1 |
| n-Pentyl esters | −12.3 | −17.4 | −6.4 | −10.8 |
| 2-Ethylhexyl esters | −4.8 | −8.0 | −3.5 | −6.1 |
| iso-Nonyl esters | −3.4 | −5.8 | −2.2 | −3.8 |
| 2-Propylheptyl esters | — | — | −2.1 | −3.7 |

TABLE 27
Loss of mass in the course of activated carbon storage
in % by mass (mixtures based on linseed oil)

| | “Non-depleted” | | “Depleted” | |
|---|---|---|---|---|
| | mixture L | | mixture L | |
| | 3 days | 7 days | 3 days | 7 days |
| Methyl esters | −14.4 | −20.8 | −12.0 | −18.5 |
| Butyl esters | −8.9 | −13.7 | −5.1 | −9.8 |
| n-Pentyl esters | −7.4 | −11.9 | −4.2 | −8.1 |
| 2-Ethylhexyl esters | −3.1 | −5.4 | −2.3 | −4.2 |
| iso-Nonyl esters | −2.5 | −4.4 | −1.7 | −3.1 |
| 2-Propylheptyl esters | — | — | −1.5 | −2.9 |

A distinct reduction in loss of mass was found, corresponding to a distinctly lower volatility of the “depleted” mixtures. This was an advantageous property for high-temperature applications and products with planned long lifetimes.

### Example 21

### Plasticizer Migration after 4 Weeks

A test specimen punched out of the pressed plaques of Example 19 (100×100 mm, thickness 1 mm) was stored at 23+/−1° C. for 24 h and weighed. The test specimen was placed between two adsorbent contact sheets such that the axes thereof coincided and a “sandwich” composite was formed. This composite was weighted down in the middle with a weight of 2 kg. The stack was positioned on a grid in the preheated temperature control cabinet (70° C.±1° C.).

The test specimen was weighed after 4 weeks. A double determination (2 test specimens per ester mixture) was conducted. The adsorbent contact sheets used were high-impact polystyrene (HIPS, thickness: 2 mm) and rigid PVC (thickness: 2 mm).

TABLE 28
Migration after 4 weeks in % by mass (mixtures based on soya oil)

| | “Non-depleted” | | “Depleted” | |
|---|---|---|---|---|
| | mixture S | | mixture S | |
| | HIPS | Rigid PVC | HIPS | Rigid PVC |
| Methyl esters | 25.3 | 21.8 | 23.1 | 19.1 |
| Butyl esters | — | — | — | — |
| n-Pentyl esters | 26.7 | 18.7 | 23.5 | 18.7 |
| 2-Ethylhexyl esters | 21.7 | 11.7 | 19.6 | 14.6 |
| iso-Nonyl esters | 21.9 | 10.7 | 20.3 | 13.7 |
| 2-Propylheptyl esters | — | — | 18.4 | 12.2 |

TABLE 29
Migration after 4 weeks in % by mass
(mixtures based on linseed oil)

| | “Non-depleted” | | “Depleted” | |
|---|---|---|---|---|
| | mixture L | | mixture L | |
| | HIPS | Rigid PVC | HIPS | Rigid PVC |
| Methyl esters | 23.6 | 21.3 | 18.0 | 20.6 |
| Butyl esters | 22.0 | 19.0 | 16.5 | 18.3 |
| n-Pentyl esters | 21.8 | 18.9 | 16.2 | 17.7 |
| 2-Ethylhexyl esters | 19.0 | 17.9 | 14.2 | 14.4 |
| iso-Nonyl esters | 17.5 | 13.6 | 14.6 | 14.2 |
| 2-Propylheptyl esters | — | — | 12.4 | 11.7 |

Inventive plasticizers show a much lower tendency to migrate in impact-modified polystyrene. This property was advantageous in the formulation of multilayer systems.

European patent application EP14182190.0 filed Aug. 26, 2014, is incorporated herein by reference.

Numerous modifications and variations on the present invention are possible in light of the above teachings. It is therefore to be understood that within the scope of the appended claims, the invention may be practiced otherwise than as specifically described herein.

1. A mixture (1), comprising:
an epoxidized fatty acid ester A; and
a compound B containing a fatty acid chain and having no functional group including a multiple bond in the fatty acid chain;
wherein a proportion by mass of compound B is smaller in said mixture (1) than a proportion of compound B in a mixture (2) of compounds containing a fatty acid chain from which the mixture (1) comprising epoxidized fatty acid ester A has been prepared.

2. The mixture (1) according to claim 1, wherein the mixture (2) from which the mixture (1) has been prepared was obtained from a vegetable oil or from a mixture of a plurality of vegetable oils.

3. A process for preparing a mixture (1) comprising an epoxidized fatty acid ester, comprising:
1) reducing a proportion of a fatty acid or fatty acid ester which does not have any functional group including a multiple bond in the fatty acid chain in a mixture (2) comprising a compound having a fatty acid chain prior to the epoxidation of the fatty acid and/or fatty acid ester;
OR
reducing a proportion of a fatty acid or fatty acid ester which does not have any functional group including a multiple bond in the fatty acid chain in a mixture (2) comprising a compound having a fatty acid chain after the epoxidation of the fatty acid and/or fatty acid ester;
2) expoxidizing said fatty acid and/or said fatty acid ester, to obtain an expoxidized fatty acid and/or an epoxidized fatty acid ester;
3) esterifying the epoxidized fatty acid, to obtain an epoxidized fatty acid ester; and
4) optionally, transesterifying said epoxidized fatty acid ester.

4. The process according to claim 3, comprising:
a) obtaining a fatty acid mixture and/or fatty acid ester mixture,
b) reducing the proportion of the fatty acid and/or the fatty acid ester which do not have any functional group including a multiple bond in the fatty acid chain in the mixture from step a),
c) epoxidizing the mixture from step b),
d) esterifying or optionally transesterifying the mixture from step c).

5. The process according to claim 3, comprising:
a) obtaining a fatty acid mixture or fatty acid ester mixture,
b) epoxidizing the mixture from step a),
c) reducing the proportion of the fatty acid or the fatty acid ester which do not have any functional group including a multiple bond in the fatty acid chain in the mixture from step b),
d) esterifying or optionally transesterifying the mixture from step c).

6. The process according to claim 3, wherein the obtaining of the fatty acid mixture or fatty acid ester mixture is from a vegetable oil or a mixture of at least two vegetable oils.

7. The process according to claim 3, wherein the proportion by mass of the compound containing a fatty acid chain and having no functional group is reduced in the process by at least 20% compared to the mixture of the compounds containing a fatty acid chain from which the mixture comprising epoxidized fatty acid ester has been prepared.

8. The process according to claim 3, wherein the proportion by mass of the compound containing a fatty acid chain and having no functional group is reduced in the process by at least 40% compared to the mixture of the compounds containing a fatty acid chain from which the mixture comprising epoxidized fatty acid ester has been prepared.

9. The process according to claim 3, wherein the proportion by mass of the compound containing a fatty acid chain and having no functional group is reduced in the process by at least 60% compared to the mixture of the compounds containing a fatty acid chain from which the mixture comprising epoxidized fatty acid ester has been prepared.

10. The process according to claim 3, wherein the proportion by mass of the compound containing a fatty acid chain and having no functional group is reduced in the process by at least 80% compared to the mixture of the compounds containing a fatty acid chain from which the mixture comprising epoxidized fatty acid ester has been prepared.

11. A mixture, comprising:
an epoxidized fatty acid ester which has been prepared by the process according to claim 3.

12. The mixture according to claim 11, wherein the proportion by mass of the compound containing a fatty acid chain and having no functional group has been reduced by at least 20% compared to the mixture of the compounds containing a fatty acid chain from which the mixture comprising epoxidized fatty acid ester has been prepared.

13. The mixture according to claim 11, wherein the proportion by mass of the compound containing a fatty acid chain and having no functional group has been reduced by at least 40% compared to the mixture of the compounds containing a fatty acid chain from which the mixture comprising epoxidized fatty acid ester has been prepared.

14. The mixture according to claim 11, wherein the proportion by mass of the compound containing a fatty acid chain and having no functional group has been reduced by at least 60% compared to the mixture of the compounds containing a fatty acid chain from which the mixture comprising epoxidized fatty acid ester has been prepared.

15. The mixture according to claim 11, wherein the proportion by mass of the compound containing a fatty acid chain and having no functional group has been reduced by at least 80% compared to the mixture of the compounds containing a fatty acid chain from which the mixture comprising epoxidized fatty acid ester has been prepared.

16. The mixture according to claim 11, wherein an alkoxide residue of the fatty acid ester in the mixture comprising epoxidized fatty acid ester is selected from the group consisting of alkoxide residues containing 1 to 20 carbon atoms.

17. The mixture according to claim 16, wherein the alkoxide residue, aside from the alkoxide function, does not have any further functional group including a multiple bond.

18. The mixture comprising epoxidized fatty acid esters according to claim 11, wherein the mixture comprises less than 20% by mass of a compound which is not a fatty acid ester.

19. A plasticizer for a polymer, comprising: the mixture according to claim 11.

20. A method for improving the gelation of a plastisol and/or for reducing the viscosity of a plastisol and/or for reducing the glass transition temperature of a composition prepared with the mixture of claim 11 and/or for improving the thermal stability of a composition prepared with the mixture of claim 11,
said method comprising:
incorporating said mixture of claim 11 in said plastisol and/or said composition.

21. A composition, comprising:
a mixture according to claim 11; and
one or more polymers selected from the group consisting of polyvinyl chloride, a copolymer of vinyl chloride with vinyl acetate or with butyl acrylate, polyalkyl methacrylate (PAMA), polyvinyl butyral (PVB), polyurethane, a polysulphide, polylactic acid (PLA), polyhydroxybutyral (PHB) and nitrocellulose.

22. The composition according to claim 21, which contains less than 5% by mass of a phthalate-containing compound.
